(19) United States
(12) Patent Application Publication
(10) Pub. No.: US 2024/0252274 A1
(43) Pub. Date: Aug. 1, 2024

(54) In-bore positioning system

(71) Applicant: Nemo Creativity Holding B.V., Oosterbeek (NL)

(72) Inventor: Timo Joric Corman Oude Vrielink, Oosterbeek (NL)

(73) Assignee: Nemo Creativity Holding B.V., Oosterbeek (NL)

(21) Appl. No.: 18/566,677

(22) PCT Filed: Jun. 9, 2022
(86) PCT No.: PCT/NL2022/050320

§ 371 Date: Dec. 4, 2023

(30) Foreign Application Priority Data
Jun. 10, 2021 (NL) 2028425

(51) Int. Cl. A61B 90/11; A61B 17/34

(52) U.S. Cl. CPC A61B 90/11; A61B 17/3403; A61B 2017/3405

(57) ABSTRACT

A positioning system is disclosed for positioning a movable object within a bore of an apparatus. At least one track is configured to be arranged along an inner surface of the apparatus. A plurality of sliders is configured to independently slide along the at least one track, and the plurality of sliders (504) are mechanically coupled to the movable object via at least one hinged connection. At least one articulated member is connected to one of the sliders via a first connection at a first end of the articulated member, and to the movable object via a second connection at a second end of the articulated member.

### FIELD OF THE INVENTION

The invention relates to a positioning system for positioning a movable object within a bore of an apparatus. The invention also relates to a method of positioning a movable object in a bore of an apparatus.

### BACKGROUND OF THE INVENTION

In 2020 a total of 2.7 million people were diagnosed with cancer. The main method for diagnosing cancer is by taking a tissue biopsy for histopathological examination. Unfortunately, the unbeknownst misplacement of biopsy needles is a major cause for false negatives, leading to cancer being detected in a later stage and thereby decreasing the likelihood of curative cancer treatment. Instrument misplacement also affects the ability to provide adequate treatment. For example, incorrect placement of ablation needles plays a role in incomplete treatment of tumors and thus causing the recurrence of cancer. Both core biopsies and ablations are predominantly performed under ultrasonic image-guidance. Ultrasound guidance is highly dependent on the human operator due to its low resolution and single plane image. Advanced imaging modalities, such as CT and MRI systems, can be used to obtain high-quality medical images in 3 dimensions. Under CT-guidance the most common technique is to position the needle before the scan is taken and verify it by taking the scan. This method requires multiple repeated scans wherein the needle is repositioned until the desired position is finally found. Ideally, positional guidance of instruments such as ablation and biopsy needles is performed under real-time and high-resolution image guidance. Unfortunately, most CT and MRI systems have a closed bore design and require additional displays for clinicians to position instruments manually.

Surgical and interventional robots are increasingly used in the healthcare domain, starting from the first clinical use in 1985 by the UNIMATE PUMA 200 robot for brain biopsies but expanding to applications such as orthopedic, spinal, abdominal, thoracic, pulmonary and gastrointestinal applications. These robots may help to provide minimally invasive access to lesions in the body. In the aforementioned applications, imaging is an important aspect for planning, navigation and in specific cases treatment. Remarkably, very few surgical systems use a real-time imaging modality—other than direct endoscopic visualization—and the ones that do use either ultrasound or fluoroscopy, which are both limited in terms of resolution and to a single planar image. The further integration of robotics and high-quality real-time imaging is a precondition for standardization and integration of machine learning algorithms to ultimately improve the clinical outcomes of robotic therapies.

Currently there are only a few systems designed for usage inside the bore of an MRI or CT scanner. The main challenge in such systems is that compatibility with the MRI or CT scanner is needed, limiting most components of the design to non-ferrous or radiolucent materials, respectively. Plastics are a type of material that typically fulfill these needs but are limited in terms of material strength and stiffness. Due to these stringent requirements alternative methods for actuation have been explored as a method to provide non-magnetic actuation methods, such as pneumatic motors (such as U.S. Pat. No. 8,061,262 B2, U.S. Pat. No. 9,469,026 B2, US 2020/0182267 A1, US2021/0007817 A1) and force transmission mechanisms.

The configuration of mechatronic components is also essential for the workspace and the access to specific anatomical areas. US 2020/0086140 A1 discloses a parallel mechanism with integrated pneumatic actuators to position a needle for the placement of brachytherapy seeds. US 2017/0367776 A1 uses a rail on which a catheter robot is placed to translate and rotate the catheter under direct MRI-guidance. These systems are placed together on the patient bed and thus require calibration and setup time for each usage. An alternative approach are patient-mounted systems in which the patient and the robot are within the same frame of reference. U.S. Pat. No. 9,326,825 B2 is a patient-mounted robot for needle interventions in for instance arthrography. US 2021/0015558 A1 and US 2019/0314110 A1 are mounted on the patient body, in particular to the scull for neurosurgical image-guided interventions.

### SUMMARY OF THE INVENTION

It is an object of the present invention to provide an improved positioning system. According to an aspect of the invention, a positioning system is provided for positioning a movable object within a bore of an apparatus, the positioning system comprising
at least one track configured to be arranged along an inner surface of the bore;
a plurality of sliders configured to independently slide along the at least one track, wherein the plurality of sliders are mechanically coupled to the movable object via at least one hinged connection; and
at least one articulated member, wherein each articulated member is connected to one of the sliders via a first hinged connection at a first end of the articulated member, wherein each articulated member is further connected to the movable object via a second hinged connection (308) at a second end of the articulated member.

This construction provides a particularly compact and versatile tool allowing a controlled movement of an object through the space of the bore. By a coordinated movement of the plurality of independently movable sliders, the movable object may be moved in multiple directions to a desired position. In this case the clinician can directly see and correct the instruments position, leading to quicker (and thus more cost-effective) interventions with a high precision. The construction may use relatively little space in the bore.

The at least one track may be linear and oriented in a longitudinal direction parallel to a central axis of the bore. This allows to easily move the movable object through the length of the bore, and still allowing radial movements by moving sliders towards or away from each other. Also, it allows an unobtrusive stand-by mode by moving the sliders away from each other, to align the members and movable object with the track.

Each articulated member may have an invariable length in between the slider and the movable object. This way, the construction can remain light-weight or relatively simple. By virtue of the sliding mechanism, the movable object can be moved through space in multiple directions.

For example, at least two of the sliders can be moved away from each other to move the movable object towards the inner surface of the bore. This way the movable object can be moved aside, so that neither the track nor the object would obstruct access to the bore.

The bore may comprise a support member for supporting an object to be subjected to the operation of the apparatus on one side of the support member, and the at least one track may be located on the same side of the support member as a space for the object to be subjected to the operation. This provides a favorable configuration in which the movable object may be moved towards or away from the object on the support member.

For example, the support member may be located in a first half of a cross section of the bore and the at least one track may be arranged in a second half of the bore opposite the first half of the cross section of the bore.

The at least one track may be suspended to at least one suspension element. This allows the track to be flexibly positioned.

The apparatus may comprise the suspension element. This facilitates the integration of the positioning system with the apparatus.

The at least one suspension element may be configured to be supported by an external object except the apparatus. This allows to provide the positioning system as a mechanically independent system, independent of the apparatus.

The at least one suspension element may comprise at least one further track oriented differently from the at least one track, wherein at least one of the at least one track is configured to slide along the at least one further track. This provides additional possibilities for moving the sliders: along the track and in the direction of the further track.

The system may comprise at least one actuator configured to move the at least one of the at least one track along the at least one further track. This provides an automated way of moving the track. This makes it easier to move the track, and thereby any sliders on the track, in order to position the movable object.

The at least one further track may have a shape corresponding to at least part of circumference of a cross section of the bore. This way, the track can be moved tangentially along the inner wall of the bore.

The wall of the bore may comprise at least one of said at least one further track. This provides an integration of the track with the apparatus.

At least one of said at least one further track may be positioned outside the bore. This allows the positioning system to be operable with minimal interference within the bore.

The system may comprise at least one actuator configured to move at least one first slider of the plurality of sliders along the at least one track independently of at least one second slider of the plurality of sliders. This facilitates the automation of the positioning system. By allowing the actuator(s) to move each slider independently, the movable object may be moved and/or rotated in any direction within the three dimensional space inside the bore.

The actuator may be mechanically coupled to the slider by means of a mechanical coupling member arranged along the at least one track. This allows the actuator to be kept relatively far away from the slider and relatively far away from the movable device, optionally even outside the bore, thus reducing interference within the bore.

The system may comprise a control unit configured to automatically control the at least one of the sliders via the at least one actuator based on a desired position or desired orientation of the movable object. The control unit thus facilitates automation of the positioning system.

The apparatus may comprise a medical apparatus, in particular a medical imaging apparatus or a radiotherapy apparatus. The inventor considered that the positioning system is particularly suited for the bore of a medical imaging apparatus or radiotherapy apparatus. In this case the clinician can directly see and correct the instrument position, which may lead to more efficient interventions and/or a higher precision. It may provide an enabling technology for robotics that may offer the ability to maneuver instruments, for example, from the clinician's console while displaying the incoming imaging data.

The movable object may comprise an end-effector or a slot for attaching an end-effector. This allows the movable object to provide an effect at a desired location.

The end-effector may comprise a transceiver or a medical device, such as a biopsy instrument, a surgical instrument, or an applicator for applying a substance. This kind of end-effector may be tedious to position in a bore during an intervention or scan, but using the positioning system this may be done more safely and/or efficiently.

At least one of the articulated members may be configured to be removably connected to the one of the sliders or the movable object, so that the at least one of the articulated members or the movable object may be replaced. This facilitates cleaning. Moreover, by temporarily removing these items, more space may be available in the bore while the positioning system is not in use.

According to another aspect of the invention, a method of positioning a movable object within a bore of an apparatus is provided, the method comprising
arranging at least one track along an inner surface of the bore;
providing a plurality of sliders on the at least one track, wherein the plurality of sliders are mechanically coupled to the movable object via at least one hinged connection;
providing at least one articulated member, wherein each articulated member is connected to one of the sliders via a first hinged connection at a first end of the articulated member, wherein each articulated member is further connected to the movable object via a second hinged connection at a second end of the elongated member; and
controlling each of the plurality of sliders to independently slide along the at least one track.

The person skilled in the art will understand that the features described above may be combined in any way deemed useful. Moreover, modifications and variations described in respect of the system may likewise be applied to the method and to the computer program product, and modifications and variations described in respect of the method may likewise be applied to the system and to the computer program product.

### BRIEF DESCRIPTION OF THE DRAWINGS

In the following, aspects of the invention will be elucidated by means of examples, with reference to the drawings. The drawings are diagrammatic and may not be drawn to scale. Throughout the drawings, similar items may be marked with the same reference numerals.

FIG. 1 shows a prior art medical imaging apparatus.

FIG. 2 shows an example of a positioning system.

FIG. 3A shows another example of a positioning system.

FIG. 3B shows a diagram of an alternative example of a positioning system.

FIG. 4 shows another example of a positioning system.

FIG. 5 shows another example of a positioning system.

FIG. 6 shows another example of a positioning system.

FIG. 7 shows a frontal view of the apparatus, provided with an example of a positioning system.

FIG. 8 shows a frontal view of the apparatus, provided with another example of a positioning system.

FIG. 9 shows a frontal view of the apparatus, provided with another example of a positioning system.

FIG. 10 shows a track suspension.

FIG. 11 shows a section view of a part of a positioning system and apparatus.

FIG. 12 shows a section view of an apparatus with a track and suspensions.

FIG. 13 shows a section view of the apparatus with another example positioning 25 system.

FIG. 14 shows a section view of the apparatus with another example of a positioning system.

FIG. 15 illustrates a cross section of a detail of the positioning system as shown in FIG. 11.

FIG. 16 shows an alternative example actuator.

FIG. 17 shows another alternative example actuator.

FIG. 18 shows a different embodiment of the articulated member.

FIG. 19 shows a functional diagram of the positioning system.

FIG. 20 shows a flowchart of a method of positioning an object within a bore of an apparatus.

### DETAILED DESCRIPTION OF EMBODIMENTS

Certain exemplary embodiments will be described in greater detail, with reference to the accompanying drawings.

The matters disclosed in the description, such as detailed construction and elements, are provided to assist in a comprehensive understanding of the exemplary embodiments. Accordingly, it is apparent that the exemplary embodiments can be carried out without those specifically defined matters. Also, well-known operations or structures are not described in detail, since they would obscure the description with unnecessary detail.

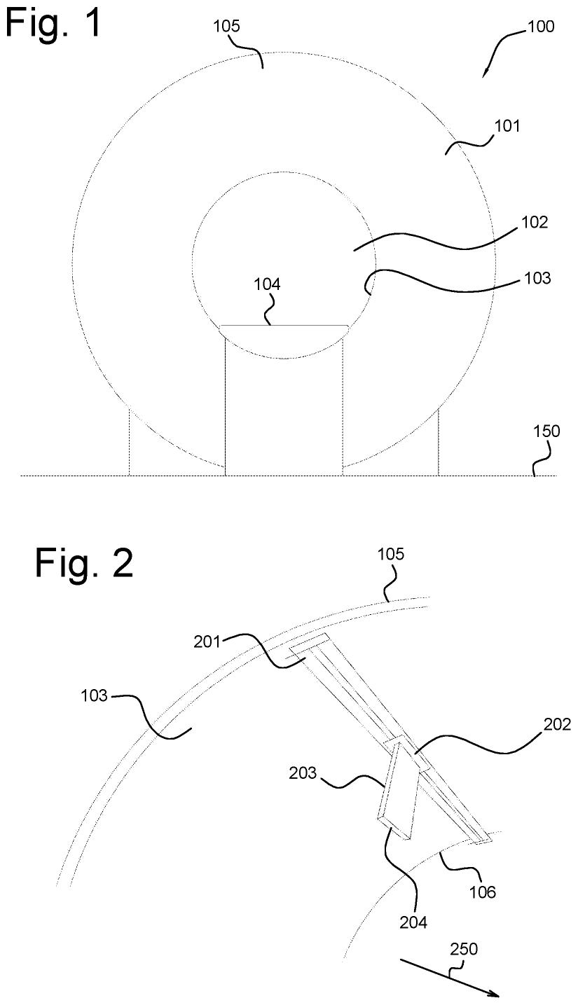

FIG. 1 shows a frontal view of a medical imaging apparatus 100. The medical imaging apparatus 100 has a body 101 with a bore 102. The body 101 may comprise, for example, a housing that encloses generators and/or detectors of certain electromagnetic fields. The apparatus 100 may comprise further components, such as a control panel, that are not illustrated. The bore 102 is bounded by the inner wall 103 of the body 101 of the apparatus 100. The bore 102 may extend from the front side 105 of the apparatus body 101 through to the back side 106 (see e.g. FIG. 2) of the apparatus body 101. In many such apparatuses, a support 104, such as a patient table, is provided on which an object to be imaged can rest inside the bore 102. The support 104 is optional and can be movable or even removable.

Although the principles of the present disclosure will be shown in relation to a medical imaging apparatus that has a bore, the same principles may be applied to other apparatuses with a bore, such as a radiation treatment apparatus or a non-medical imaging apparatus. Moreover, although the images show an apparatus with a horizontally oriented bore and horizontal patient support, this is not a limitation. The techniques disclosed herein may also be applied to apparatuses with a vertical or oblique oriented bore. The support may be e.g. a vertical support plate to which an object may lean. Also, the principles may be applied to apparatuses with a through bore, i.e. a bore with openings on both ends of the bore. Alternatively, the principles of the present disclosure may be applied to a bore in the form of a cavity that has an opening on only one end, wherein the bore is an e.g. cylindrical space that is closed on one end and open on the other end. In yet other applications of the techniques disclosed herein, such as ‘open MRI’, the bore may be closed on only one side and open on three sides. The bore 102 may be generally cylindrical in shape with a longitudinal axis (usually extending from one opening of the bore to another opening of the bore) and a radius.

The apparatus 100 may rest on a floor 150. However, this is not a limitation, The apparatus 100 may alternatively suspend from a ceiling or have a support construction in any other way.

FIG. 2 illustrates an example of a positioning system for positioning a movable object inside the bore 102 of the apparatus 100. The positioning system comprises a track 201, a slider 202 configured to slide along the track, and a member 203 fixed on one end to the slider 202, wherein the movable object (not illustrated) can be attached to the other end 204 of the member 203. The member 203 may extend from the inner wall 103 into the bore 102. The track 201 may be oriented in a longitudinal direction 250 of the bore. In certain embodiments, the track 201 may extend from the front side 105 of the body 101 to the back side 106 of the body 101. For example, the track 201 may be arranged on, in, or close to the inner wall 103 of the apparatus 100. The illustrated configuration allows moving the movable object in the longitudinal direction. To move the object in radial direction or to rotate the movable object, other means would be necessary in addition to the track 201 and slider 202.

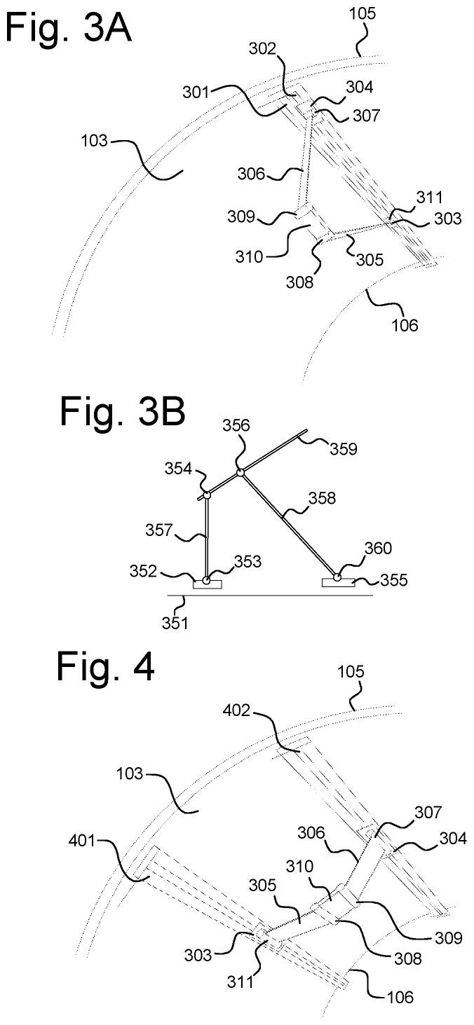

FIG. 3A illustrates a second example of a positioning system for positioning a movable object inside the bore 102 of the apparatus 100. The positioning system comprises two tracks 301 and 302, one adjacent to the other. On the first track 301, there is a first slider 303. On the second track 302, there is a second slider 304. A first end of a first elongated member 305 is attached to the first slider 303 by a hinge 311. A first end of a second elongated member 306 is attached to the second slider 304 by a hinge 307. A platform 310 is attached to the second end of the first elongated member 305 and the second end of the elongated member 306. The first elongated member 305 is attached to the platform 310 by a hinge 308, and the second elongated member 306 is attached to the platform 310 by a hinge 309, wherein there is a distance between the hinge 308 and the hinge 309. By moving the sliders 303 and 304 along their track, the position of the platform 310 may be changed; the platform 310 may be moved in longitudinal or radial direction, depending on the movement of the sliders 303 and 304. The platform 310 thus is a movable object that can be moved by independently sliding the sliders 303 and 304. The platform 310 may alternatively be a platform for another movable object. By simply attaching an object on the platform 310, a movable object may be created. For example, an end-effector in a medical procedure may be attached to the platform 310 to become the movable object.

In certain embodiments, the hinges are optional. That is, not all connections between articulated members 306 and the sliders 304 need to be hinged. In certain embodiments, at least one articulated member 306 is rigidly connected to the slider 304 and hinged connected to the movable object or platform 310. In certain embodiments, at least one articulated member 306 is hinged connected to the slider 306 and rigidly connected to the movable object or platform 310.

FIG. 3B shows a diagram of such a configuration. The figure shows slider 351 with two sliders 352 and 355. The first slider 352 is connected to a first end of elongated member 357, which is not hinged to the slider 352, but is connected to the slider 352 by a rigid connection 353, such as integrated molding or a bond. On the other end, opposite the first end, the elongated member 357 has a hinged connection 354 to the movable object 359. Articulated member 358 has a hinged connection 360 to the second slider 355 and a hinged connection 356 to the movable object 359. In this way, an at least one-dimensional translation movement of the movable object 359 is possible, in addition to a rotational movement.

In the example of FIG. 3A, two adjacent tracks 301 and 302 are shown, one track for each slider. This may facilitate the independent movement by actuators, as will be discussed elsewhere in the present disclosure. However, that is not a limitation. Other embodiments may use a single track for multiple sliders, using an independent actuator for each slider, so that each slider can slide independent from the other sliders along the track. In certain embodiments with multiple sliders on the same track, such sliders cannot cross each other.

In the example shown in FIG. 3A, the tracks 301, 302 are oriented longitudinally along the inner wall 103. However, this is not a limitation. In certain embodiments, for example, the tracks may be arc shaped, arranged tangentially along the inner wall

FIG. 4 shows an example of a positioning system, in which two tracks 401 and 402 are spaced further apart compared to the example of FIG. 3A. This may provide additional possibilities in moving the movable object 310. The details of the hinging attached members 305 and 306 and the movable object 310 is similar to the configuration shown in FIG. 3A, and is not repeated here for conciseness.

Although the illustration shows hinges with one degree of freedom, this is not a limitation. Other kinds of hinges, such as hinges with two or three degrees of freedom, such as spherical joints, or ball joints, may be applied alternatively. In certain embodiments, compliant joints may be used as the hinged connections, using a compliant mechanism, which is a flexible mechanism that achieves force and motion transmission through elastic body deformation. Compliant joints may have the advantage of reduced friction and/or the possibility to achieve a relatively large positioning accuracy.

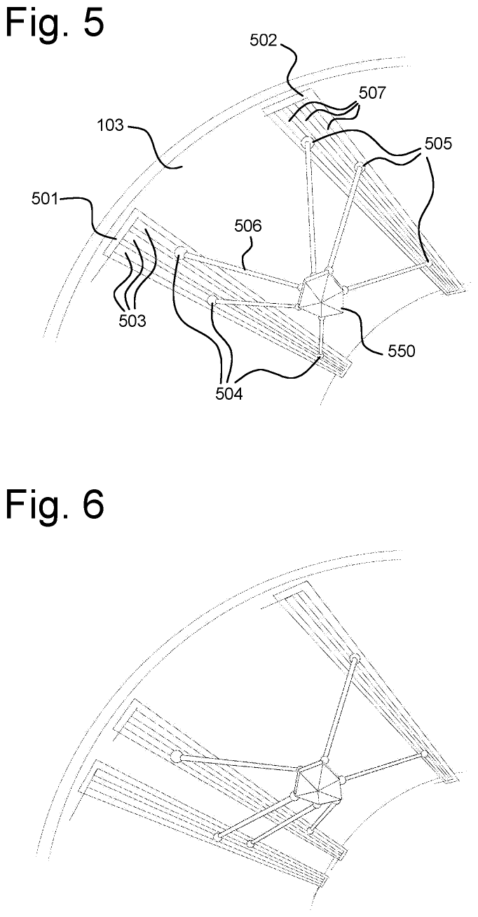

FIG. 5 shows another example of a positioning system, in which two spaced-apart multi-slider tracks 501, 502 are provided. The two spaced-apart multi-slider tracks 501, 502 are oriented in longitudinal direction of the bore 102 and located on the inner surface 103 of the apparatus 100. Each multi-slider track 501, 502 has a plurality of sliders. The sliders are connected to a movable object 550 by articulated members 506. In the shown example, the multi-slider track 501 comprises a plurality of adjacent parallel tracks 503. Similarly, the multi-slider track 502 comprises a plurality of adjacent parallel tracks 507. These adjacent parallel track 503, 507 may be implemented in the form of rails, for example. Each slider 504 is arranged to slide along a different track 503 of the multi-slider track 501. Each slider 505 is arranged to slide along a different track 507 of the multi-slider track 502. The sliders 504, 505 are connected to the movable object 550 by means of respective articulated members 506, with a hinging connection on both ends of the articulated members 506. The articulated members 506 are connected at different positions on the movable object 550, allowing to control the orientation of the movable object 550.

In an alternative embodiment, a multi-slider track 501, 502 may be implemented as a single track (e.g. a rail) on which a plurality of sliders may be mounted.

In an alternative embodiment, curved parallel tracks, e.g. multi-slider tracks, may be implemented with tangential orientation instead of longitudinal orientation with respect to a central axis of the bore 102.

FIG. 6 shows another configuration of a positioning system for positioning a movable object in a bore of an apparatus. The system comprises three multi-slider tracks. In the illustrated configuration, on each multi-slider track, two sliders are mounted. The distance between the tracks can vary.

In general, it may be advantageous that the tracks are parallel. In certain embodiments, where the tracks are not arranged in parallel, they may be arranged, preferably so that they do not cross each other.

Although several configurations of tracks and sliders have been shown in the illustrations, these illustrations merely serve to show examples, but are not limiting. Other configurations of the tracks are also considered. The illustrations of FIGS. 2 to 6 show tracks that may be fixed with respect to each other and with respect to the wall of the bore. For example, this may be realized by attaching the tracks to the wall of the bore, for example by means of adhesion. Alternatively, the tracks may be integrated in the wall of the bore. For example, the tracks may be implemented in a slit in the wall of the bore. Yet alternatively, the tracks may be suspended, for example by suspension constructions at the openings of the bore. As will be shown in the following, such a suspension construction may allow to move the track as necessary.

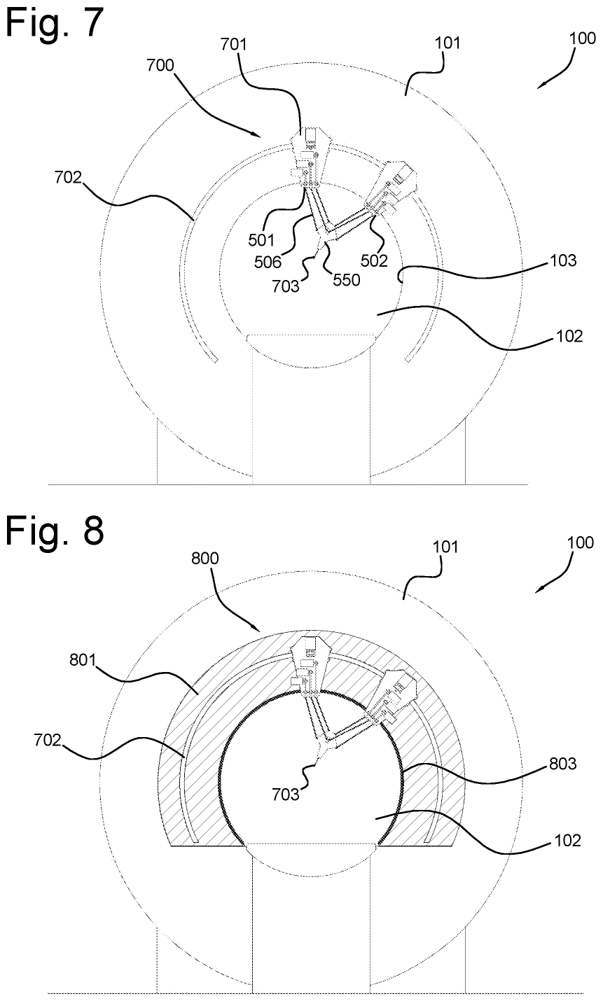

FIG. 7 shows a frontal view of the apparatus 100, provided with an example of a positioning system 700. The positioning system 700 may be understood best in combination with FIG. 5. The tracks 501, 502 of the positioning system are positioned in the bore 102, longitudinally along the inner wall 103 of the apparatus 100 (perpendicular to the viewing direction of FIG. 7). Moreover, the tracks 501 are suspended on a track suspension 701. The track suspension 701 is in turn, attached to a curved track 702 by means of a slider, so that the track suspension 701 can slide along the curved track 702. This way, by sliding the suspension 701 along the curved track 702, the tangential position of the track 501 inside the bore 102 can be changed. Some or all of the tracks 501 inside the bore 102 can be made movable in this way. Suspension track 702 and suspensions 701 may be provided on both ends of the bore 102, to provide stability to the track 510. The suspension track 702 may be integrated in the body 101 of the apparatus 100. Alternatively, the suspension track 702 may be attached to the body 101 of the apparatus 100 by fixation means, such as adhesion or screws. Yet alternatively, the suspension track 702 may be held in place by any suitable means.

As illustrated in FIGS. 7, 8, 9, 12, 13, and 14, and end-effector 703 may be mounted on the movable object 550. This end-effector may be integrated with the movable object 550. Alternatively, the movable object 550 may contain a slot in which the end-effector 703 may be (removably) inserted. Other ways to mount the end-effector 703 on the movable object 550 can be considered in view of the present disclosure. The end-effector 703 may be positioned by the positioning system 100, for example to perform an intervention, e.g. take a sample or inject a fluid.

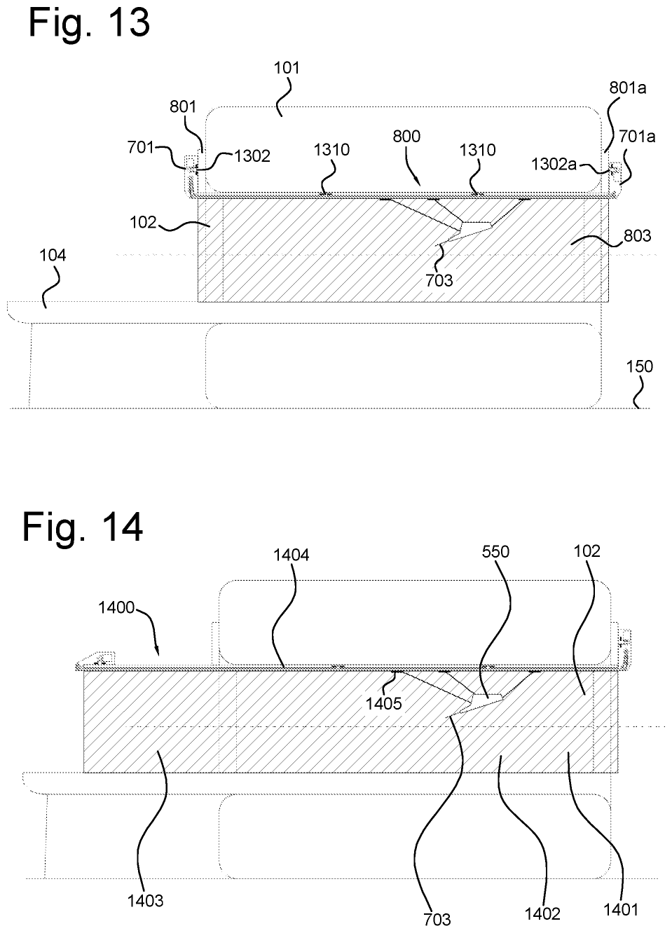

FIG. 8 shows a frontal view of the apparatus 100, provided with an example of a positioning system 800. The positioning system 800 is similar to positioning system 700. However, the positioning system 800 further comprises a front mounting plate 801, on which the suspension track 702 is mounted. Moreover, the inner surface 103 of the apparatus body 101 is partly covered by a liner 803. This will be further elucidated hereinafter with reference to FIG. 13.

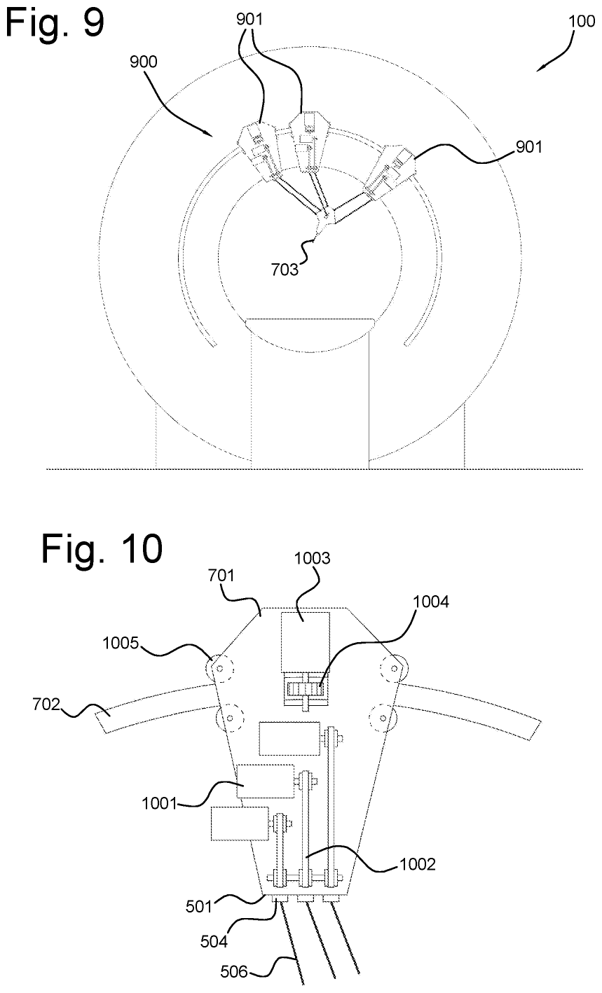

FIG. 9 shows a frontal view of the apparatus 100, provided with an example of a positioning system 900. The positioning system 900 has three tracks with two sliders each, and may therefore be understood in combination with FIG. 6. The construction and operation of the positioning system 900 is, apart from the number of tracks, similar to the positioning system 700. In the example shown in FIG. 9, there are three sliding track suspensions 901, one for each track.

FIG. 10 shows one of the track suspensions 701 in greater detail. The tracks 501 extend in a direction substantially orthogonal to the circular track 702. Moreover, the sliders 504 may be operatively connected to actuators 1001. These actuators may be implemented in several ways. For example, each slider 504 may be connected, by means of a wire or belt 1002, to an actuator 1001 on both ends of its track 501. The actuator 1001 may be configured to pull the belt 1002, in order to move the slider closer to it. That way, in certain embodiments by activating an actuator on either side of the track 501, the slider 504 may be moved in either direction. By moving the slider, a force is exerted on the corresponding articulated member 506, and thus the movable object 550 is moved. Moreover, the suspension 701 itself may be moved along the track 702, by a further actuator 1003. For example, the actuator 1003 may actuate a rack-and-pinion mechanism 1004, which can move the suspension 701 in both directions along the circular track 702. Guiding wheels 1005 may be provided for further stability, to keep the suspension 701 well aligned with the circular track 702.

It is noted that suitable types of actuators may include actuators that are compatible with the operation of the apparatus 100. In case the apparatus 100 comprises an MRI apparatus, the actuators may preferably be MRI compatible actuators. Such MRI compatible actuators may include piezo motors, pneumatic drivers, and hydraulic motors. It helps that the actuators may be kept out of the bore. In case of a CT apparatus, for example, a stepper motor could be used outside the bore.

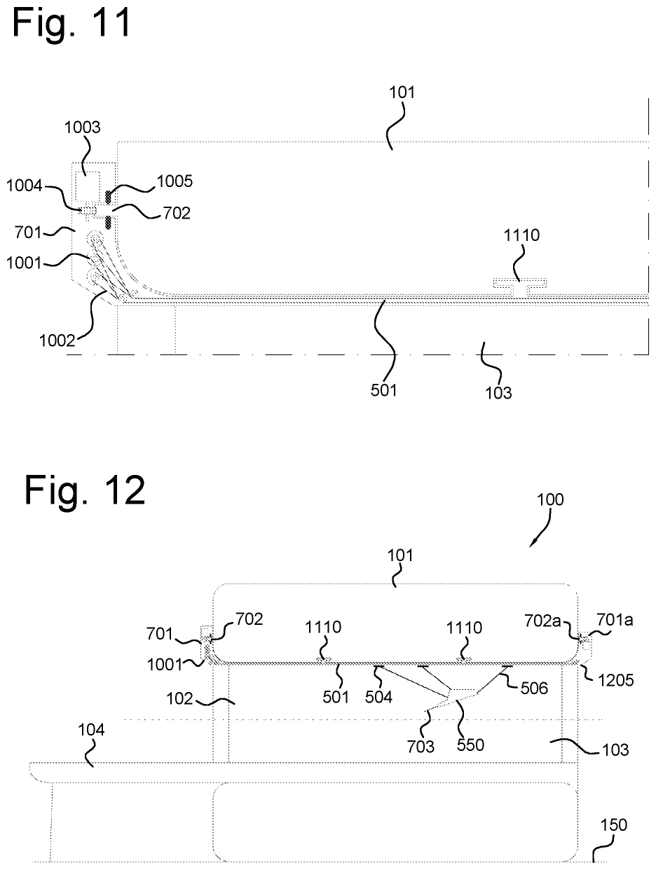

FIG. 11 shows a section view of a part of the body 101 and inner wall 103 of the apparatus 100. The Figure shows the track 501 and the suspension 701 that can move along the circular track 702. The construction is similar to the construction discussed above with respect to e.g. FIGS. 5, 7, and 10. As shown in FIG. 11, the track 501 may be reinforced in between its two ends by a connection to the inner wall 103. This reinforcement may have the form of a supporting slot or guide 1110 in the inner wall 103. The slot or guide 1110 is another track along which the track 501 may slide, when the suspensions 701 on both ends of the track 501 are moved. The slot or guide 1110 may help to prevent the track 501 from bending due to gravity or other (reactive) forces transmitted through the articulated members, for example.

FIG. 12 shows a section view of an apparatus 100 with a track 501 and suspensions 701, 701a that can slide on tracks 702, 702a on both ends of the track 501. On the track 501, there are the sliders. Also shown is the movable object 550 connected to the sliders 504 through the articulated members. Although only one of the tracks 501 is visible, it will be understood that in practical implementations there may be more than one track 501. In certain embodiments, as shown in FIG. 12, the actuators 1001 for the sliders 504 are provided on one end of the track 501, in suspension 701. On the other end of the track 501, in suspension 701a, idler pulleys 1205 are provided. This way, the slider 504 can be moved in both directions with a single actuator 1001. A mechanism (not illustrated) may further be provided to keep a sufficient tension on the belt 501.

FIG. 13 shows a section view of the apparatus 100 with the positioning system 800. As mentioned above with reference to FIG. 8, the positioning system 800 comprises a front mounting plate 801 and a back mounting plate 801a, one mounting plate on each end of the bore 102. Moreover, the positioning system 800 further comprises a liner 803 on the wall of the bore. The liner 803 may be made, for example, of a sturdy material such as a synthetic material. Preferably the liner 803 is made of a material that is compatible with the operation of the apparatus 100, for example MRI-compatible material or a material that is radiolucent, i.e., transmissive for radiation (e.g. X-rays) that is emitted and/or detected by the apparatus 100. For example, the liner 803 may be regarded as a tube inside the bore 102. Examples of suitable material are engineering plastics, such as methyl methacrylate (PMMA), polyetheretherketone (PEEK), polyoxymethylene (POM), carbon-fiber composites, nylon, or non-ferrous metals with a low magnetic susceptibility, such as titanium, tungsten, platinum or a suitable alloy.

It is noted that a liner 803 is only an example of a manner to add the positioning system to an existing apparatus 100. Another implementation could provide, for example, a frame instead of a solid liner.

The liner 803 and front and back mounting plates 801, 801a may act as a construction to provide mechanical stability to the positioning system 800 independently of the construction of the apparatus 100. That is, the tracks 1302, 1302a for the suspensions 701, 701a, may be part of the front and back mounting plates 801, 801a, and the supporting tracks 1310 inside the bore may be part of the liner 803. The front and back mounting plates 801, 801a may be attached to the liner 803 by use of molding or fasteners. Fasteners, such as nylon screws, may have the advantage that it is easier to assemble and disassemble the positioning system.

For example, the liner 803 may have a horseshoe shape in cross section, the ends resting on the apparatus 100 inside the bore 102, for example on both sides of the support 104.

FIG. 14 shows a section view of the apparatus 100 with an example of a positioning system 1400. The positioning system 1400 is largely similar to the positioning system 800. However, the liner 1401 with the tracks 1404 of the positioning system 1400 extends beyond the length of the bore 102. This way, the sliders 1405 can slide out of the bore 102 along their track 1404. This may be helpful in certain cases where the movable object 550 needs to be moved outside of the bore 102, or close to the outside of the bore 102.

In certain embodiments, the liner 1401 and the tracks 1404 are extendable. That is, the liner 1401 can be made shorter or longer, to extend in length beyond the length of the bore 102. For example the liner 1401 comprises two halves 1402, 1403 that engage with each other in a telescopic fashion, to create a longer liner 1401 or a shorter liner 1401. For example, in certain implementations, the lines that connect the actuators with the sliders may be sufficiently long, and may be provided with e.g. an idler pulley, so that they can support the extended length of the liner 1401.

It will be understood that different implementations of an extended length positioning system are possible. It is not necessary to make the whole liner 1401 longer. Alternatively, it is possible to extend just the tracks beyond the end of the bore 102, for example in a telescopic fashion. This way, the sliders 1405 can move along the track 1404 outside the bore 102.

In yet another embodiment, the whole positioning system 800 or 1400 may be movable with respect to the apparatus 100. For example, the liner 803, 1401 can be shifted in longitudinal direction through the bore 102.

In certain embodiments of the positioning system in general, the members 506 that connect the movable object 550 to the sliders 504 has a variable and controllable length, for example based on a piston or a linear actuator. This provides more flexibility in the positioning, although the mechanical complexity increases.

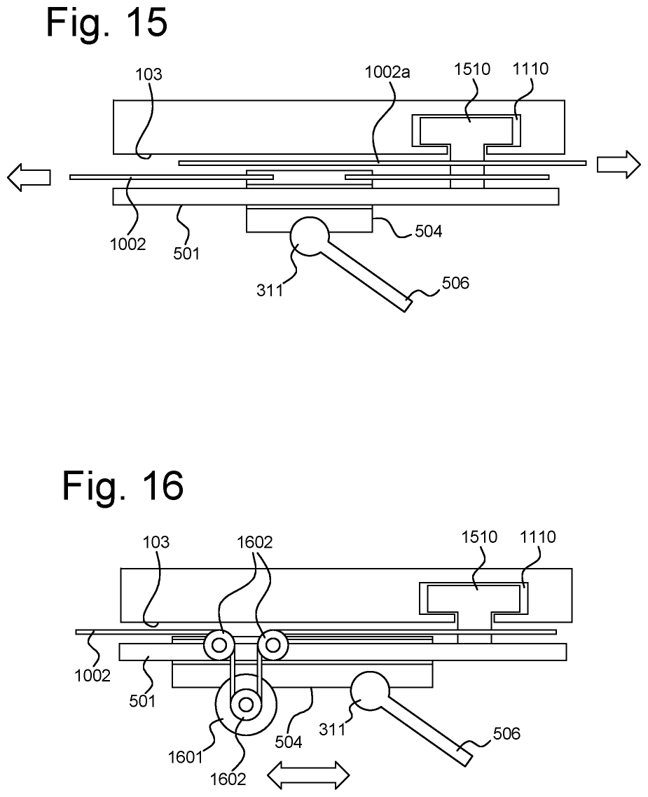

FIG. 15 illustrates a cross section of a detail of the positioning system as shown in FIG. 11, including a slider 504 with articulated member 506 and hinged connection 311. As shown, a track 501 can be implemented as, for example, a rail or a bar or a rigid tube, to which the slider 504 is engaged. Also, the track 501 may comprise an extension 1510 that engages with a slot 1110 in the inner wall 103 (or alternatively in the liner 803), to keep the track 501 aligned with the inner wall 103 (or liner 803) along its length. To move the slider 504 along the track 501, and/or to keep the slider 504 in a particular place, lines 1002 may connect the slider 504 to actuators 1001 of suspensions 701, 701a. To allow multiple individually movable sliders on the same track 501, separate lines 1002, 1002a may be provided for each slider 504.

FIG. 16 shows an alternative for the actuators built in suspensions 701, 701a. In the alternative configuration of FIG. 16, an actuator 1601 is built into the slider 504. The actuator and pulleys 1602 can cause the slider 504 to move along the track 501. This configuration allows to employ a plurality of sliders 504 on a single track 501 with a single line 1002.

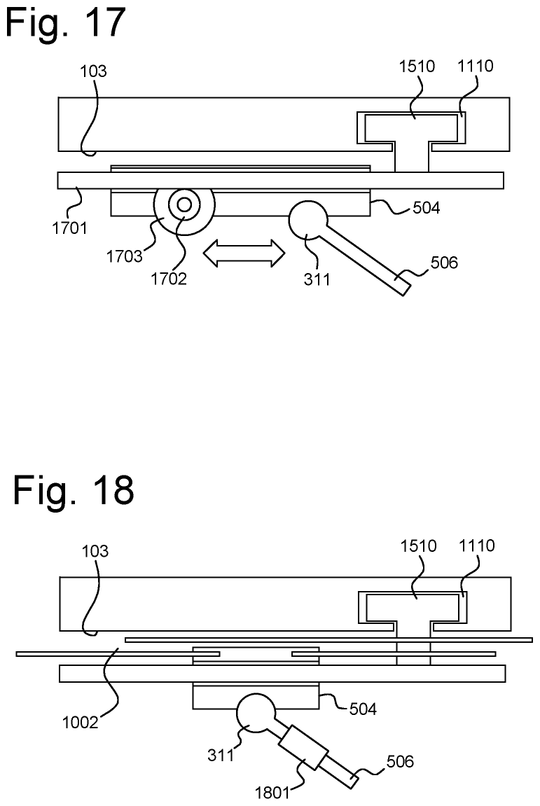

FIG. 17 shows another alternative for the actuators built in suspensions 701, 701a. In the alternative configuration of FIG. 17, an actuator 1703 is built into the slider 504. The track 1701 is provided with a rack. The actuator 1703 and pinion 1702 can cause the slider 504 to move along the track 501 by rotating the pinion 1702. This configuration allows to employ a plurality of sliders 504 on a single track 501 without using a line 1002.

FIG. 18 shows a different embodiment of the articulated member 506. In this configuration, the articulated member 506 comprises a force sensor 1801. This provides a haptic feedback to the positioning system. The force sensor 1801 may comprise, for example, a Wheatstone bridge, which can measure a force by detecting a change in electrical resistance) or a fiber based optical deformation detection, such as a fiber brag. The fiber based force sensor may have a better compatibility with e.g. MRI. It is noted that the force sensor 1801 may be implemented as an additional feature of any embodiment of the positioning system.

In certain embodiments, the articulated members are rigid, elongated members. For example, the articulated members may be rods. In another example, the articulated members may be rectangular strips of a rigid material. Other shapes of the articulated members are alternatively possible.

In certain embodiments, the articulated members/rods may be removed and replaced with (different) ones. For example, the articulated members may be connected to the sliders and/or the movable object by means of a clicking connection system. In certain embodiments, the movable object may be removable from the articulated members.

The positioning system disclosed herein may be useful for providing a flexible positioning system suitable for example for robotic interventions, while being less disruptive and easy to fold away when not in use or while positioning an object, such as a patient, in the bore, before such an intervention. The lean construction makes the positioning system particularly suitable for MRI compatibility. Also, the construction causes relatively little disruption of electromagnetic fields, including radio waves and/or any electromagnetic signals generated or detected by apparatuses such as magnetic resonance imaging (MRI), computed tomography (CT), radiotherapy, and the like.

The sliding mechanism may allow the forces to be distributed over several articulated members. The sliding mechanism also helps the accuracy, because positional errors may be averaged out rather than accumulated. Since the forces are distributed over the articulated members, these forces can be relatively low. That allows less stiff materials to be used. Since MRI compatible materials may be generally less stiff, this property allows to optimize the positioning system for MRI compatibility. Thus, the positioning system may be able to combine a relatively high positioning accuracy with a relatively little weight of construction and relatively low forces exerted in the system. Moreover, the positioning system can be designed, as illustrated herein, with actuators outside the imaging area (e.g., outside the bore), thus providing more available space inside the bore, and reduced effect of materials and electric currents.

FIG. 19 shows a functional diagram of the positioning system 1900. The positioning system 1900 comprises a control unit 1901, which may comprise, for example, one or more computer processors and memory. The memory may, for example, store computer readable instructions that cause the processor to perform the steps set forth herein. The memory may further store a spatial model of the positioning system. The control unit 1901 may be configured to control the operation of the positioning system 1900. To that end, the positioning system 1900 may also comprise a user interface unit 1902, which may comprise a display and an input unit such as a touch screen or a keyboard. In certain embodiments, the user interface unit 1902 may comprise a haptic controller. Haptic feedback may be generated, for example, based on forces detected by force sensors 1801. Alternatively, the positioning system 1900 may comprise a communication unit 1903, such as a wireless or wired network connection, to connect the positioning system 1900 to an integrated computer system, in order to allow the positioning system 1900 to be controlled or programmed by an external device, such as a picture and archiving system (PACS), or a console of the apparatus 100. Alternatively, the communication unit 1903 may be configured to receive information, such as images that can be used to control the movements of the movable object, from such an external device. The positioning system further comprises a plurality of actuators 1904, as described hereinabove. The memory of the control unit 1901 may store a spatial model of the tracks, sliders, movable object, and actuator modes. For example, the control unit 1901 may be configured to receive a target location and orientation for the movable object 550, via the user interface unit 1902 or the communication unit 1903. The control unit 1901 may further be configured to map the target location and orientation to target positions of the sliders 504. This mapping may be done by means of the spatial model. The control unit 1901 may be further configured to translate the target positions of the sliders into command signals for the actuators 1904. The control unit 1901 may be configured to transmit the command signals to the actuators 1904.

In certain embodiments, the positioning system 1900 further comprises one or more sensors 1905. The sensors 1905 may, for example, comprise a force sensor 1801 configured to detect a force exerted to an articulated member 506. In case a force is detected, the control unit 1901 may be configured to generate an alarm signal and/or stop the actuators 1904. Alternatively, forces detected by force sensors 1801 in the articulated members 506 may be combined, using the spatial model, to estimate the forces on the movable object 550. The sensors 1905 may further comprise a sensor that can detect the current position of the sliders and/or of the movable object, which may allow the control unit 1901 to further optimize the control signals sent to the actuators 1904. Yet alternatively, the position of the movable object may be detected by analyzing an image generated by the apparatus 100.

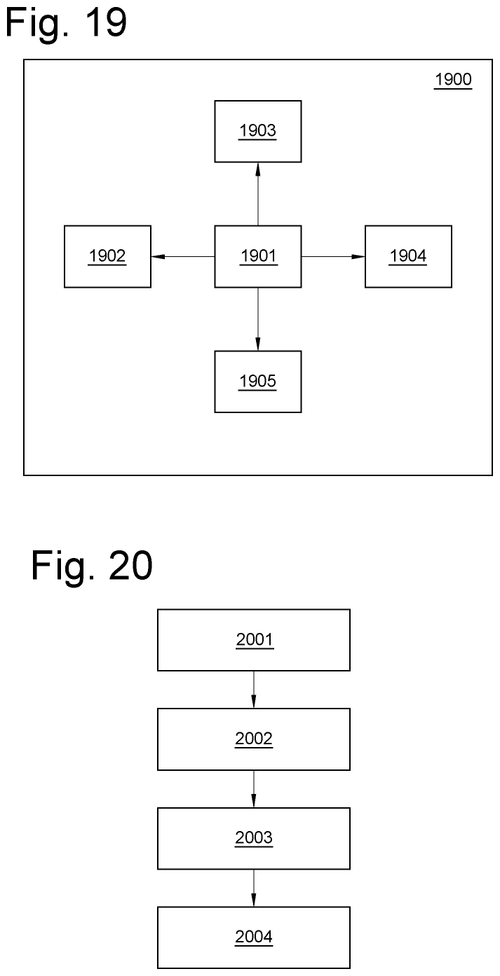

FIG. 20 shows a flowchart of a method of positioning an object within a bore of an apparatus. The method comprises step 2001 of arranging at least one track along an inner surface of the bore. The method further comprises step 2002 of providing a plurality of sliders on the at least one track. The method further comprises step 2003 of providing a plurality of articulated members inside the bore, wherein each articulated member is connected to one of the sliders via a first connection at a first end of the articulated member, wherein each articulated member is further connected to the movable object via a second hinge connection at a second end of the elongated member. The method further comprises step 2004 of controlling each of the plurality of sliders to independently slide along the at least one track.

An alternative use of the in-bore positioning system is for applying substances into the body, in particular for providing in-situ in-vivo bio-printing of substances and tissues for use for regenerative medicine. The application of hydrogels or other biocompatible scaffolding material can be also used for treatment of spina bifida during fetal surgery. In certain embodiments, the end-effector of the movable object may be a tool for this purpose, such as an in-situ 3D printer.

Some or all aspects of the invention may be suitable for being implemented in form of software, in particular a computer program product. The computer program product may comprise a computer program stored on a non-transitory computer-readable media. Also, the computer program may be represented by a signal, such as an optic signal or an electromagnetic signal, carried by a transmission medium such as an optic fiber cable or the air. The computer program may partly or entirely have the form of source code, object code, or pseudo code, suitable for being executed by a computer system. For example, the code may be executable by one or more processors.

The examples and embodiments described herein serve to illustrate rather than limit the invention. The person skilled in the art will be able to design alternative embodiments without departing from the spirit and scope of the present disclosure, as defined by the appended claims and their equivalents. Reference signs placed in parentheses in the claims shall not be interpreted to limit the scope of the claims. Items described as separate entities in the claims or the description may be implemented as a single hardware or software item combining the features of the items described.

Certain aspects are defined in the following clauses.

1. A positioning system for positioning a movable object (550) within a bore (102) of an apparatus (100), the positioning system comprising
at least one track (501) configured to be arranged along an inner surface (103) of the apparatus (100);
a plurality of sliders (504) configured to independently slide along the at least one track (501), wherein the plurality of sliders (504) are mechanically coupled to the movable object (550) via at least one hinged connection (354); and
at least one articulated member (506), wherein each articulated member (506) is connected to one of the sliders (504) via a first hinged connection (311) at a first end of the articulated member (506), wherein each articulated member (506) is further connected to the movable object (550) via a second hinged connection (308) at a second end of the articulated member (506).

2. The system of clause 1, wherein the at least one track (401) is linear and is oriented in a longitudinal direction (250) parallel to a central axis of the bore (102).

3. The system of clause 1, wherein each articulated member (506) has an invariable length in between the slider (504) and the movable object (550).

4. The system of clause 1, wherein at least two of the sliders (303, 304) can be moved away from each other to move the movable object (310) towards the inner surface (103) of the apparatus (100).

5. The system of clause 1, wherein the bore (102) comprises a support member (104) for supporting an object to be subjected to the operation of the apparatus (100) on one side of the support member (104), and the at least one track (501) is located on the same side of the support member (104) as a space for the object to be subjected to the operation.

6. The system of clause 1, wherein the at least one track (501) is suspended to at least one suspension element (701).

7 The system clause 6, wherein the apparatus (100) comprises the at least one suspension element.

8. The system of clause 6, wherein the at least one suspension element (701) is configured to be supported by an external object (150) except the apparatus (100) or by a bottom surface of the bore 102.

9. The system of clause 6, wherein the at least one suspension element (701) comprises at least one further track (702) oriented differently from the at least one track (501), wherein at least one of the at least one track (501) is configured to slide along the at least one further track (702).

10. The system of clause 9, further comprising at least one actuator (1003) configured to move the at least one of the at least one track (501) along the at least one further track (702).

11. The system of clause 9, wherein the at least one further track (702) has a shape corresponding to at least part of a circumference of a cross section of the bore (102).

12. The system of clause 11, wherein the wall (103) of the bore (102) comprises at least one of said at least one further track (1110).

13. The system of clause 11, wherein at least one of said at least one further track (1302) is positioned outside the bore (102).

14. The system of clause 1, further comprising at least one actuator (1001) configured to move at least one first slider (504) of the plurality of sliders along the at least one track (501) independently of at least one second slider (1204) of the plurality of sliders.

15. The system of clause 14, wherein the at least one actuator (1001) is mechanically coupled to the slider (504) by means of a mechanical coupling member (1002) arranged along the at least one track (501).

16. The system of clause 14, further comprising a control unit (1901) configured to automatically control the at least one of the sliders (504) via the at least one actuator (1001) based on a desired position or desired orientation of the movable object (550).

17. The system of clause 1, wherein the apparatus (100) comprises a medical apparatus, in particular a medical imaging apparatus or a radiotherapy apparatus.

18. The system of clause 1, wherein the movable object (550) comprises an end-effector (1350) or a slot for attaching an end-effector (1350)

19. The system of clause 18, wherein the end-effector (1350) comprises a transceiver or a medical device, such as a biopsy instrument, a surgical instrument, or an applicator for applying a substance.

20. The system of clause 1, wherein at least one of the at least one articulated member (506) is configured to be removably connected to the one of the sliders (504) or the movable object (550), so that the at least one of the at least one articulated member (506) or the movable object (550) may be replaced.

21. A method of positioning a movable object within a bore of an apparatus, the method comprising
arranging (2001) at least one track along an inner surface of the bore;
providing (2002) a plurality of sliders on the at least one track, wherein the plurality of sliders are mechanically coupled to the movable object via at least one hinged connection;
providing (2003) at least one articulated member, wherein each articulated member is connected to one of the sliders via a first hinged connection at a first end of the articulated member, wherein each articulated member is further connected to the movable object via a second hinged connection at a second end of the articulated member; and
controlling (2004) each of the plurality of sliders to independently slide along the at least one track.

1. A positioning system for positioning a movable object within a bore of an apparatus, the positioning system comprising
at least one track configured to be arranged along an inner surface of the bore;
a plurality of sliders configured to independently slide along the at least one track, wherein the plurality of sliders are mechanically coupled to the movable object via at least one hinged connection;
at least one articulated member, wherein each articulated member is connected to one of the sliders via a first hinged connection at a first end of the articulated member, wherein each articulated member is further connected to the movable object via a second hinged connection at a second end of the articulated member; and
at least one actuator configured to move at least one first slider of the plurality of sliders along the at least one track independently of at least one second slider of the plurality of sliders.

2. The system of claim 1, wherein the at least one track is linear and is oriented in a longitudinal direction parallel to a central axis of the bore.

3. The system of claim 1, wherein each articulated member has an invariable length in between the slider and the movable object.

4. The system of claim 1, wherein at least two of the sliders can be moved away from each other to move the movable object towards the inner surface of the bore.

5. The system of claim 1, wherein the bore comprises a support member for supporting an object to be subjected to the operation of the apparatus on one side of the support member, and the at least one track is located on the same side of the support member as a space for the object to be subjected to the operation.

6. The system of claim 1, wherein the at least one track is suspended to at least one suspension element.

7. The system claim 6, wherein the apparatus comprises the at least one suspension element.

8. The system of claim 6, wherein the at least one suspension element is configured to be supported by an external object except the apparatus or by a bottom surface of the bore.

9. The system of claim 6, wherein the at least one suspension element comprises at least one further track oriented differently from the at least one track, wherein at least one of the at least one track is configured to slide along the at least one further track.

10. The system of claim 9, further comprising at least one actuator configured to move the at least one of the at least one track along the at least one further track.

11. The system of claim 9, wherein the at least one further track has a shape corresponding to at least part of a circumference of a cross section of the bore.

12. The system of claim 11, wherein the wall of the bore comprises at least one of said at least one further track.

13. The system of claim 11, wherein at least one of said at least one further track is positioned outside the bore.

14. (canceled)

15. The system of claim 1, wherein the at least one actuator is mechanically coupled to the slider by means of a mechanical coupling member arranged along the at least one track.

16. The system of claim 1, further comprising a control unit configured to automatically control the at least one of the sliders via the at least one actuator based on a desired position or desired orientation of the movable object.

17. The system of claim 1, wherein the apparatus comprises a medical apparatus, in particular a medical imaging apparatus or a radiotherapy apparatus.

18. The system of claim 1, wherein the movable object comprises an end-effector or a slot for attaching an end-effector.

19. The system of claim 18, wherein the end-effector comprises a transceiver or a medical device, such as a biopsy instrument, a surgical instrument, or an applicator for applying a substance.

20. The system of claim 1, wherein at least one of the at least one articulated member is configured to be removably connected to the one of the sliders or the movable object, so that the at least one of the at least one articulated member or the movable object may be replaced.

21. A method of positioning a movable object within a bore of an apparatus, the method comprising
arranging at least one track along an inner surface of the bore;
providing a plurality of sliders on the at least one track, wherein the plurality of sliders are mechanically coupled to the movable object via at least one hinged connection;
providing at least one articulated member, wherein each articulated member is connected to one of the sliders via a first hinged connection at a first end of the articulated member, wherein each articulated member is further connected to the movable object via a second hinged connection at a second end of the articulated member; and
controlling each of the plurality of sliders to independently slide along the at least one track.
